(19) United States
(12) Patent Application Publication
(10) Pub. No.: US 2023/0347062 A1
(43) Pub. Date: Nov. 2, 2023

(54) DEVICE FOR ADMINISTERING A LIQUID TO A HUMAN BY MEANS OF INJECTION, COMBINATION OF SUCH A DEVICE AND AN INJECTOR COMPRISING AN INDIVIDUAL DOSE OF A LIQUID AND AN INJECTION NEEDLE, AND METHOD FOR MANIPULATING AN INJECTOR

(71) Applicant: STEMBERG BV, Wijnegem (BE)

(72) Inventors: Koen Catharina Lodewijk BEYERS, Wuustwezel (BE); Vanessa Vicky Jill VANKERCKHOVEN, Wilrijk (BE)

(73) Assignee: STEMBERG BV, Wijnegem (BE)

(21) Appl. No.: 18/042,795

(22) PCT Filed: Sep. 1, 2021
(86) PCT No.: PCT/EP2021/074145

§ 371 Date: Feb. 24, 2023

(30) Foreign Application Priority Data
Sep. 2, 2020 (BE) 2020/5607

(51) Int. Cl. A61M 5/31; A61M 5/315

(52) U.S. Cl. CPC A61M 5/3137; A61M 5/31591

(57) ABSTRACT

A device for administering a liquid to a human by means of injection of liquid. The device is configured with a movement device arranged to move a gripping element for gripping an injector. The injector is adapted with an individual dose of the liquid and an injection needle and preferably comprises a housing and an operating member to expel the liquid from the injector via the injection needle. The gripping element includes a first component for engaging the housing and a second component for operating an operating member. The first component and the second component are resistively slidable relative to each other, and the first component is connected to the movement device via the second component.

### CROSS REFERENCES TO RELATED APPLICATIONS

This Non-Provisional patent application claims the benefit of and priority to PCT Application Serial No. PCT/EP2021/074145, filed Sep. 1, 2021, entitled “DEVICE FOR ADMINISTERING A LIQUID TO A HUMAN BY MEANS OF INJECTION, COMBINATION OF SUCH A DEVICE AND AN INJECTOR COMPRISING AN INDIVIDUAL DOSE OF A LIQUID AND AN INJECTION NEEDLE, AND METHOD FOR MANIPULATING AN INJECTOR,” which claims the benefit of and priority to Belgian Patent Application Serial No. 2020/5607, filed Sep. 2, 2020, bearing the same title as the PCT application, the entire contents of both application which are hereby incorporated herein by reference.

### BACKGROUND

The present invention concerns a device for administering a liquid to a human by means of injection, a combination of such a device and an injector comprising an individual dose of a liquid and an injection needle, and a method for manipulating an injector.

In particular, the invention relates to such a device which is adapted to automatically administer the liquid to a human, using single-use injectors for injection, the injectors each containing a needle and an individual supply, in other words a supply intended for one individual, of the liquid, for example a vaccine.

In particular, such injectors are adapted for intradermal and/or subcutaneous injection. This means that they are adapted to insert an injection needle into a human body only to a certain desired depth corresponding to the injection depth for intradermal or subcutaneous injection.

In particular, such injectors are adapted to administer an injection with a very limited number of operations that do not require any special training or experience.

Such injectors are known, for example from WO2013/156524, WO2017/168015, WO2016/117164A1 and U.S. Pat. No. 8,066,680B2. Nevertheless, such injectors require a person to manipulate them and to operate them in order to effect the injection, in other words administer the liquid.

### SUMMARY

The invention aims to reduce the number of persons required for administering the liquid and to increase reproducibility of administering the liquid, and therefore concerns a device for administering a liquid to a human by means of injection of the liquid, the device comprising a movement device which is arranged to move a gripping element for gripping an injector, the injector comprising an individual dose of the liquid and an injection needle.

Preferably the gripping element is movable between different spatial positions by means of the movement device. A movement device is considered to be any device capable of automatically moving an object, in this case an injector, between different spatial positions relative to a fixed point of the movement device, whereby preferably intended movements of the movement device are programmable, whereby preferably the movement device is arranged to pick up and release such an object at predefined spatial positions and to do so repeatedly.

An example and a preferred embodiment of such a movement device is a robotic arm.

Due to such a device according to the invention, an injection with the liquid can be administered easily and rapidly and in a reproducible fashion to a large numbers of people. This has the further advantage that little or no contact is required between healthcare personnel and said people. Also, the surface of such a device is easy to disinfect manually or automatically. Both of these factors help to reduce the risk of transmission of infectious viruses, such as SARS-CoV-2, if the liquid has to be administered to a large number of people, as is necessary, for example, in large-scale vaccination campaigns.

In a preferred embodiment, said movement device has a movable section which is arranged to move, preferably between different positions, the movable section being provided with said gripping element.

In a preferred embodiment, said movement device is arranged to move said gripping element between different positions.

In a preferred embodiment, said movement device is a robotic arm and said movable section is an end of said robotic arm, wherein said end is arranged to move between different positions, the end being provided with said gripping element.

By a robotic arm is meant a movable mechanical arm provided with one or more drives allowing it to move without human intervention based upon signals received by the one or more drives from a control unit which is optionally programmable.

A robotic arm is not intended to mean a device for sensing the movement of and enhancing the functioning of an arm of a human. Preferably the robotic arm is an articulated robot.

In a preferred embodiment the different spatial positions comprise a first position for gripping an injector by the gripping element, an injection position for operating the injector and a second position for removing the injector from the gripping element

In a preferred embodiment the device is arranged to effect gripping an injector by the gripping element autonomously and to effect removing an injector from the gripping element autonomously, whereby autonomously means without human intervention aside from starting up the device and providing an injector for gripping by the gripping element.

In a preferred embodiment, the device comprises a positioning unit for positioning and temporarily fixing a limb to be injected of a human body. This allows the administration of the liquid to take place in a reliable and reproducible manner, because the limb into which the administration is to be done, is always in the same position.

Preferably, the positioning unit comprises a first clamping member with a first surface and a second clamping member with a second surface, the second surface being movable relative to the first clamping member in a direction at least partially perpendicular to the first surface to thereby clamp the limb between the first clamping member and the second clamping member.

Preferably, the first surface is located above the second surface.

In a preferred embodiment, the second clamping member is an air cushion. As a result, a limb can be temporarily fixed in a reproducible, reversible, rapid and automated manner, whereby the load on the limb, and therefore the risk of injury or discomfort, is very limited.

In a preferred embodiment, the positioning unit comprises a passage for passing or letting through the injector or a part of the injector. In this case, the positioning unit preferably has an activated state, i.e. a state in which the limb is fixed, in which part of the outer surface of the limb is pushed by the positioning unit against a surface surrounding an end of the passage. As a result, the part of the limb to be injected is always in exactly the same spatial position, such that the injection can be performed in a reliable way.

In this case, the surface surrounding the end of the passage preferably forms part of the first surface. Also preferably, the passage is located in the first clamping member.

In a preferred embodiment, the limb is an arm with a hand, the positioning unit comprising a hand rest for the hand.

In a preferred embodiment, the device is adapted to place the injector on the limb.

In a preferred embodiment, the device is arranged to operate the injector, i.e. to cause the liquid to move out of the injector via the injection needle, and more preferably the gripping element is arranged to operate the injector.

In a preferred embodiment, the device is adapted to place the injector on the limb by means of a straight linear movement of a movable section of the movement device and/or of the gripping element and to operate it by continuing the same straight linear movement of the movable section and/or the gripping element, the linear movement preferably being a downward movement. The injection can hereby be administered in an easy manner and with a limited number of controllable actuators.

In a preferred embodiment, the injector comprises a housing and an operating member to cause the liquid to move out of the injector via the injection needle, the gripping element being provided with a first component for engaging the housing, the gripping element being provided with a second component for operating the operating member, wherein the first component and the second component are resistively slidable relative to each other, preferably in the direction of said linear movement, the first component being connected to the movement device only via the second component.

As a result, the gripping element can be both placed and operated by means of a single linear movement. Because of the resistance in the movement of the first component and second component relative to each other, a first part of the linear movement will cause the housing to be moved such that the injector is correctly positioned, ie against the limb to be injected, without the risk that the operating member is operated. Once the housing is correctly positioned, a further movement of the housing is restricted, for example by the limb to be injected, such that the resistance is overcome and the operating member is operated during a second part of the linear movement.

In a preferred embodiment, the device comprises a supply station for a supply of injectors and a receiving station for receiving used injectors, wherein gripping element is adapted to move from the supply station to an application position, also called an injection position, i.e. the position where the liquid is administered to a person, and subsequently to the receiving station, wherein the gripping element is adapted to receive an injector from the supply station, wherein the device is adapted to release a used injector from the gripping element at the receiving station.

This allows the device to perform and repeat all the operations required to inject the liquid without human intervention.

In a preferred embodiment, the robotic arm is a robotic arm with at least 5 axes, the axes being oriented such that the orientation of the gripping element relative to a reference point external to the device can remain constant with a spatial movement of the end of the robotic arm.

Preferably, the device is a device for intradermally or subcutaneously administering the liquid. In case the liquid is a vaccine, intradermal administration requires a smaller amount of liquid than intramuscular administration.

In a preferred embodiment, the device is provided with a means for opening a package of said injector, the package opening means being attached to the gripping element or to the end of the robotic arm, and preferably forming part of the gripping element.

As a result, injectors do not have to be removed manually and/or in advance from a protective packaging, such that the number of human actions is limited and it is prevented that more or less than the required number of injectors is removed from a protective packaging.

The invention also concerns a combination of a device according to the invention and at least one injector which comprises an individual dose of the liquid and an injection needle.

In a preferred embodiment of the device and combination, the liquid is a vaccine.

In a preferred embodiment of the device and combination, the injector comprises a housing and an operating member for causing the liquid to move out of the injector via the injection needle.

In a preferred embodiment of the device and combination, the injector has a storage state in which the injection needle is located within a protective cap and the injector has a use state in which the injection needle projects outside the protective cap, preferably wherein the injector is adapted to be brought from the storage state to the use state by placing the protective cap on a limb of a person to be injected and subsequently pushing the housing towards the protective cap.

In a preferred embodiment of the device and combination, the injector and/or the device is provided with safety means to prevent the injection needle from being inserted further than a certain depth into the limb.

In a preferred embodiment of the device and combination, the injector is an injector adapted to be used once.

In a preferred embodiment of the device and combination, the injector is adapted for intradermal or subcutaneous injection of the liquid, and preferably contains safety features that make another type of injection difficult or impossible.

Preferably, the injector is an injection device as defined in claim 14 or 15 of WO2017/168015 or an intradermal injection device as defined in any one of the claims of EP2844317B1 or an intradermal delivery device as defined in any one of the claims of US2002/045858A1.

For the sake of completeness, it is noted that the injector can be an injector with only one injection needle, but also an injector with more than one injection needle, for example two or eight injection needles, which are adapted to penetrate the skin together. This is also known as a ‘needle array’.

The invention also relates to a method for manipulating an injector, the method comprising a positioning step for positioning the injector on a limb of a person, wherein in this positioning step an injector comprising an individual dose of liquid to be injected and comprising a injection needle, is picked up by a movement device at a first position, and is moved by means of the movement device from the first position to the limb and is placed on the limb. This is an easy, rapid and inexpensive way to prepare for an injection without the need for human intervention.

In a preferred variant, after the placing step, the method comprises a removal step wherein the injector is moved by the movement device from the limb to a second position, whereby at this second position the injector is removed from the movement device. This is an easy, rapid and inexpensive way to perform post-injection operations without the need for human intervention.

In a preferred variant, before the placement step is completed, the limb is temporarily fixed by means of a positioning unit, such that injection becomes easier.

In a preferred variant, in the method for manipulating an injector, a combination according to the invention is used.

In the abovementioned method and its variants, the method excludes any medical aspect, but is limited to one or more steps taking place before injection and after removing the injection needle from the limb of the person.

Alternatively, the method according to the invention can be defined as the use of a device according to the invention for receiving an injector comprising an individual dose of liquid to be injected and comprising a injection needle and placing this injector on a limb of a person.

In a preferred variant of the use, by means of the device, the liquid is subsequently injected, preferably intradermally or subcutaneously, into the limb.

In case the liquid is a vaccine, intradermal administration requires a smaller amount of liquid than intramuscular administration.

The invention also relates to a method for injecting a liquid into a human body, wherein an injector is used which comprises an individual dose of liquid to be injected and which comprises an injection needle and which comprises an operating member for causing the liquid to move out of the injector via the injection needle, wherein the injector is picked up by a movement device and placed on a limb of a person to be injected, wherein subsequently the injection needle is inserted into the limb of the person to be injected by the movement device, wherein subsequently the control member is operated by the movement device.

Preferably, after the operating member has been operated, the movement device moves the injector from the limb to a position where the injector is removed from the movement device.

In a preferred variant, in the method for injecting a liquid into a human body, a combination according to the invention is used.

In a more limited variant, the invention can be defined according to the following clauses:

Clause 1: Device for administering a liquid to a human by means of injection of the liquid, the device comprising a robotic arm with an end arranged to move between different positions, the end being provided with a gripping element for gripping an injector, the injector comprising an individual dose of the liquid and an injection needle.

Clause 2: Device according to clause 1, wherein the device comprises a positioning unit for positioning and temporarily fixing a limb to be injected of a human body, the positioning unit comprising a first clamping member with a first surface and a second clamping member with a second surface, the second surface being movable relative to the first clamping member in a direction at least partially perpendicular to the first surface to thereby clamp the limb between the first clamping member and the second clamping member.

Clause 3: Device according to clause 2, wherein the second clamping member is an air cushion.

Clause 4: Device according to clause 2 or 3, wherein the positioning unit comprises a passage for passing the injector or part of the injector.

Clause 5: Device according to any one of the preceding clauses, which is arranged to place the injector on the limb by means of a linear movement of the end of the robotic arm and to operate it by continuing the linear movement of the end of the robotic arm, the linear movement preferably being a downward movement.

Clause 6: Device according to any one of the preceding clauses, wherein the injector is an injector comprising a housing and comprising an operating member to cause the liquid to move out of the injector via the injection needle, the gripping element being provided with a first component for engaging the housing, the gripping element being provided with a second component for operating the operating member wherein the first component and the second component are resistively slidable relative to each other, the first component being connected to the robotic arm via the second component.

Clause 7: Device according to any one of the preceding clauses, wherein the device is a device for intradermally or subcutaneously administering the liquid.

Clause 8: Device according to any one of the preceding clauses, wherein the device is provided with a means for opening a package of a said injector, the means for opening the package being attached to the end of the robotic arm, and preferably forming part of the gripping element.

Clause 9: Method for manipulating an injector, the method comprising a positioning step for positioning the injector on a limb of a person, wherein in this positioning step an injector comprising an individual dose of liquid to be injected and comprising an injection needle, is picked up by a robotic arm at a first position, and is moved by means of the robotic arm from the first position to the limb and is placed on the limb.

Clause 10: Method according to clause 9, wherein the method, after the positioning step, comprises a removal step wherein the injector is moved by the robotic arm from the limb to a second position, whereby at this second position the injector is removed from the robotic arm.

Clause 11: Method according to clause 9 or 10, wherein the limb is temporarily fixed by means of a positioning unit before the placement step is completed.

Clause 12: Method according to any of clauses 10 to 12, wherein a device according to any one of clauses 1 to 8 is used.

### BRIEF DESCRIPTION OF THE DRAWINGS

In order to better explain the invention, a preferred embodiment of a device and a combination according to the invention is described, without the invention being limited to this embodiment, with reference to the following figures:

FIG. 1 shows a perspective view of a device according to the invention;

FIG. 2 shows a top view of the device of FIG. 1;

FIG. 3 shows a component of the device of FIGS. 1 and 2;

FIG. 4 illustrates another component of the device of FIGS. 1 and 2;

FIG. 5 shows the component of FIG. 4 in cross section along V-V;

FIG. 6 shows a component of the combination according to the invention in a partially disassembled state;

FIG. 7 shows the component of FIG. 6 in its use condition;

FIG. 8 shows the component of FIG. 7 in cross section according to VIII-VIII;

FIGS. 9 to 11 show the device of FIG. 1 in three different use conditions, with a part of the housing of the device being omitted in FIG. 10;

FIGS. 12 and 13, respectively, show a front view along F11 of the part of FIG. 3, in use conditions corresponding to FIGS. 9 and 10, respectively;

FIGS. 14 and 15, respectively, show a cross section of a part of the combination according to the invention, on a larger scale and in two different use states;

FIGS. 16 to 18 show a perspective view of part of the combination according to the invention, on a larger scale and in three different use states;

The device 1 and injector 2 shown in the figures are a device and injector for intradermally injecting a liquid into a person.

### DETAILED DESCRIPTION

The device 1 mainly consists of a robotic arm 3 and an arm holder 4 for fixing a forearm of a person to be injected. Of course, the device 1 also comprises a control unit, not shown, for controlling the movements of the robotic arm 3 and of other moving parts of the device 1.

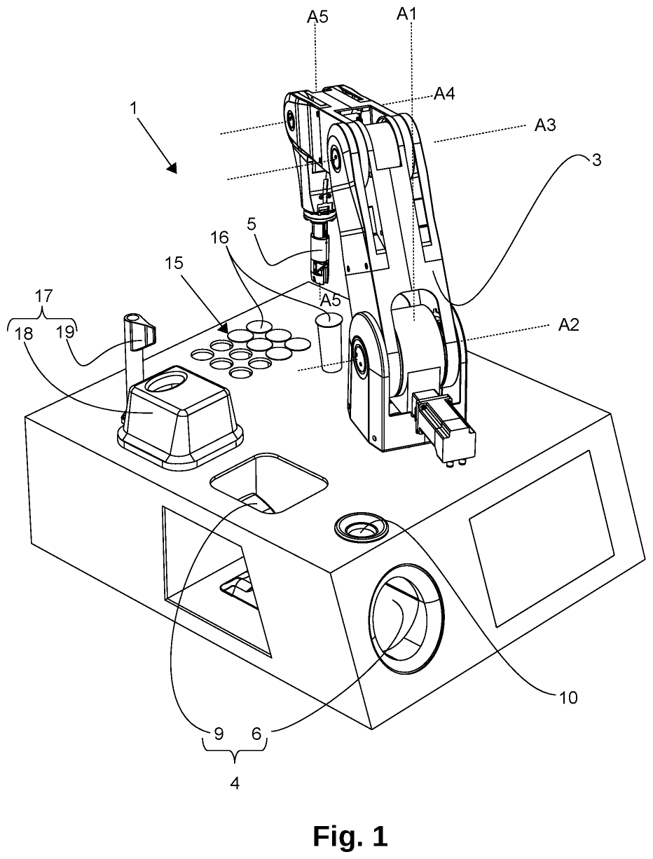

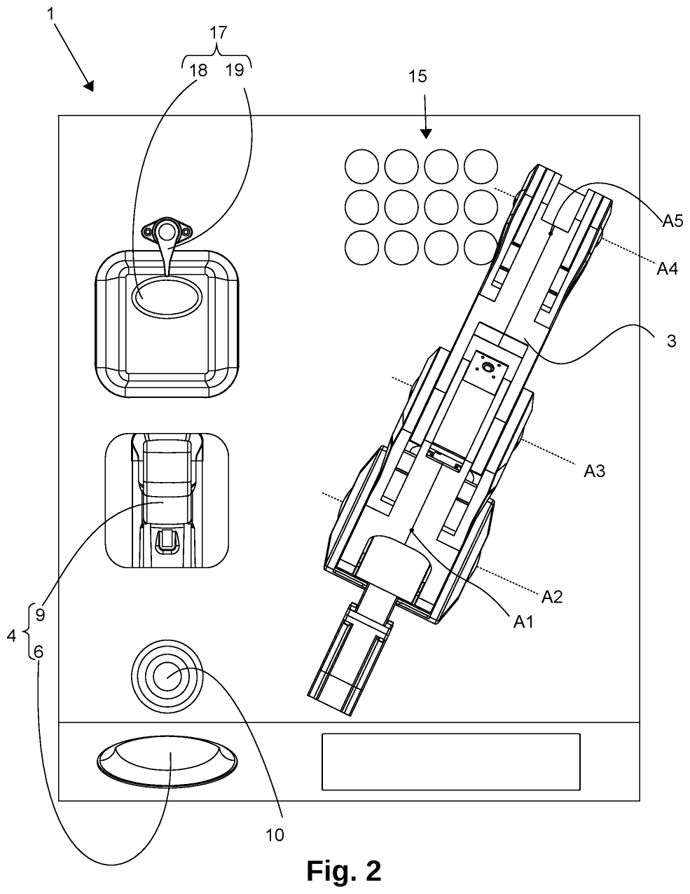

The robotic arm 3 is a five-axis robotic arm A1,A2,A3,A4,A5 as shown in FIGS. 1 and 2. At the end of the robotic arm 3 a gripping element 5 is attached for receiving and operating an automatic injector 2 for administering intradermal injections.

The robotic arm 3 with the five axes A1,A2,A3,A4,A5 is arranged in such a way that the gripping element 5 can be kept in the same orientation everywhere within the reach of the robotic arm 3, whereby the end of the robotic arm 3, and thus the attached gripping element 5, can be rotated about a vertical geometric axis A5 through that end.

The arm holder 4, shown in detail in FIG. 3, comprises a short tunnel 6 formed by an annular tunnel body 7 for receiving a forearm 8 of a person to be injected and a hand rest 9 for that person's hand. The tunnel body 7 and the hand rest 9 are spaced from each other such that when the palm of the hand is placed on the hand rest 9, a portion of the forearm 8 is located in the tunnel 6.

The tunnel body 7 has an inner surface. A vertical passage 10 for the passage of an injector 2 is arranged in the top of the tunnel body 7. This passage 10 is surrounded on its bottom by the upper part 11 of the inner surface of the tunnel body 7. An inflatable air cushion 12 is arranged in the bottom of the tunnel 6. The air cushion 12 has an upper surface 13 which, by inflating the air cushion 12, can move upwards.

The device 1 comprises a supply station 15 for injectors to be used 2. In this example, this supply station 15 is formed by overlapping holes in the housing of the device 1, wherein a package 16 with an injector 2 can be properly fitted in each of the holes. For the sake of clarity, in FIG. 1, but not in the other figures, such a package 16 is shown outside the supply station 15, it being noted that such a package 16 will not be in that location during normal use.

The device 1 comprises a receiving station 17 for used injectors 2, which mainly consists of a receptacle 18 for used injectors 2 and a specially shaped pin 19 placed above the receptacle 18, which can cooperate with the gripping element 5, as will be explained below, for releasing an injector 2 from the robotic arm 3.

Due to the five axes A1,A2,A3,A4,A5 of the robotic arm 3, the end of the robotic arm 3 can be moved, wherein the gripping element 5 with its main axis always remains vertically oriented, and wherein the gripping element 5 is able to rotate about its main axis.

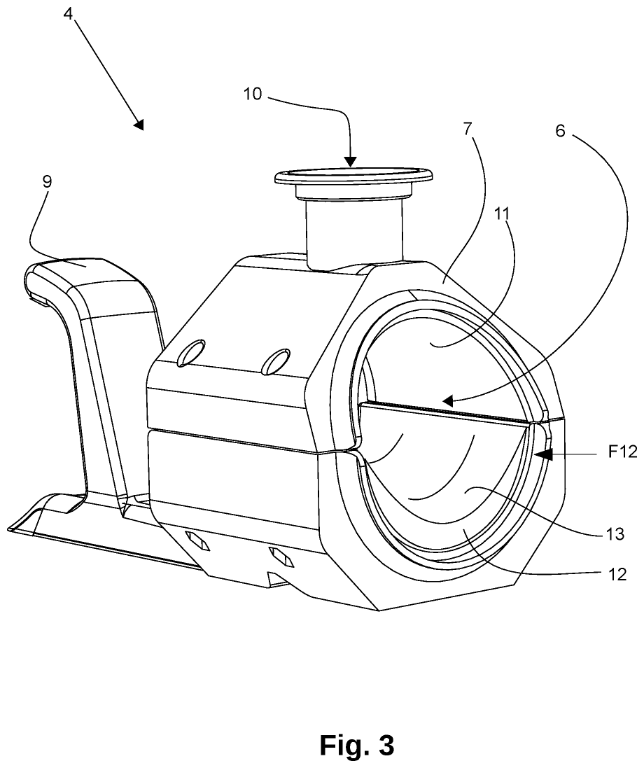

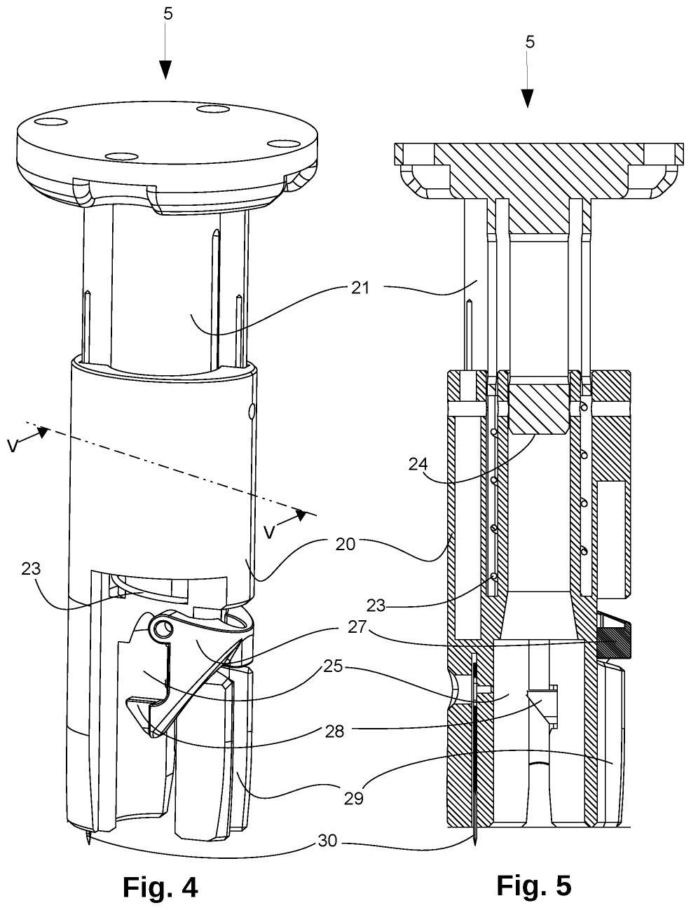

The gripping element 5, as shown in detail in FIGS. 4, 5, 14 and 15, mainly consists of a first, outer component 20 and a second, inner component 21, with which the gripping element 5 is attached to the end of the robotic arm 3. The lower part of the second component 21 is slidable in the first component 20, in vertical direction. A push spring 23 is arranged between the first component 20 and the second component 21, such that the second component 21 can only be resistively moved in the first component 20. The second component 21 is provided on its bottom with an abutment 24 for making contact with a plunger of an injector 2.

In the first component 20, a receiving cavity 25 is provided for receiving a housing 26 of an injector 2.

The first component 20 is also provided with a hook element 27 with one or more, in this example two, hooks 28 for holding an injector 2 in the gripping element 5 and with a guide groove 29 for said pin 19. The hook element 27 is spring-loaded and pivotally suspended from the first component 20 and is sprung such that the hook element 27 is maintained in the state as shown in FIG. 4. A small knife 30 is arranged on the underside of the first component 20 in order to be able to cut open a package 16 of an injector 2.

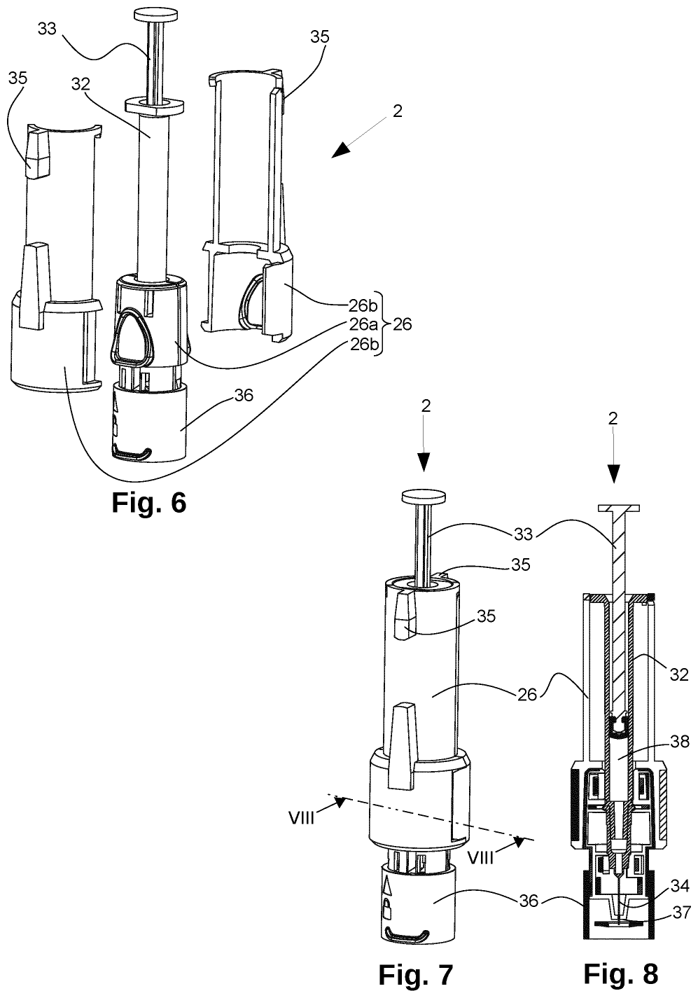

In this example, the injector 2 is, but not necessarily, an injector 2 known under the name VAX-ID of the company Novosanis in Belgium and which is intended for single use. This injector 2 is known from WO2017/168015, but the construction and operation of the injector 2 will be briefly explained for the sake of completeness. The injector 2 is shown in detail in a storage state in FIGS. 6 to 8 and 14 and in an operating state in FIG. 15.

The injector 2 mainly consists of a housing 26 in which a syringe 32 with a plunger 33 and an injection needle 34 is arranged. In this example, the housing 26 consists of the original housing 26a of the VAX-ID injector 2 and additional housing shells 26b for causing the injector 2 to cooperate with the gripping element 5. In particular, the housing 26 comprises for this purpose one or more, in this example two projections 35 on the housing 26 which cooperate with the hook element 27 when the injector 2 is coupled to the gripping element 5 in order to be able to hold the injector 2 in the gripping element 5.

The injector 2 comprises a protective cap 36 in which the injection needle 34 is located in the storage state. The protective cap 36 is resistively slidable with respect to the housing 26, in an upward direction. The distance over which the protective cap 36 and the housing 26 are movable relative to each other, is limited.

The lower portion of the housing 26 forms a flat surface 37 surrounding the injection needle 34, from which the injection needle 34 projects a short distance, in this example 0.85 mm.

The syringe 32 is prefilled with liquid to be injected 38, in this example a vaccine, for example a BCG vaccine or a rabies vaccine or a vaccine against SARS-CoV-2.

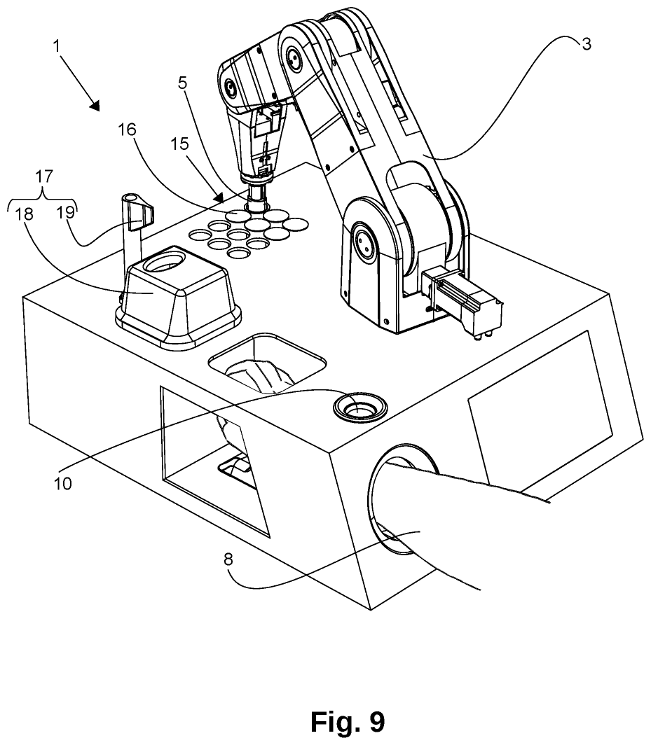

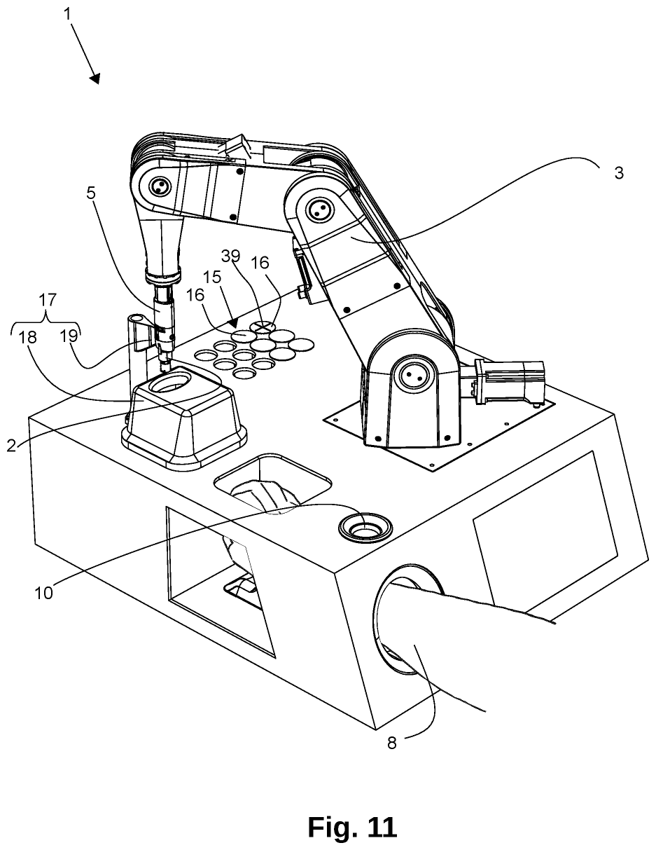

The operation of the device 1 and combination according to the invention is shown below and is shown in FIGS. 9 to 18. At the start, the air cushion 12 is not inflated, and the device 1 is in a neutral state as shown in FIG. 1.

First, a person to be injected slides his hand and forearm 8 through the tunnel 6 and places the palm of his hand on the hand rest 9. The forearm 8 of this person is located between the air cushion 12 and the top of the tunnel body 7.

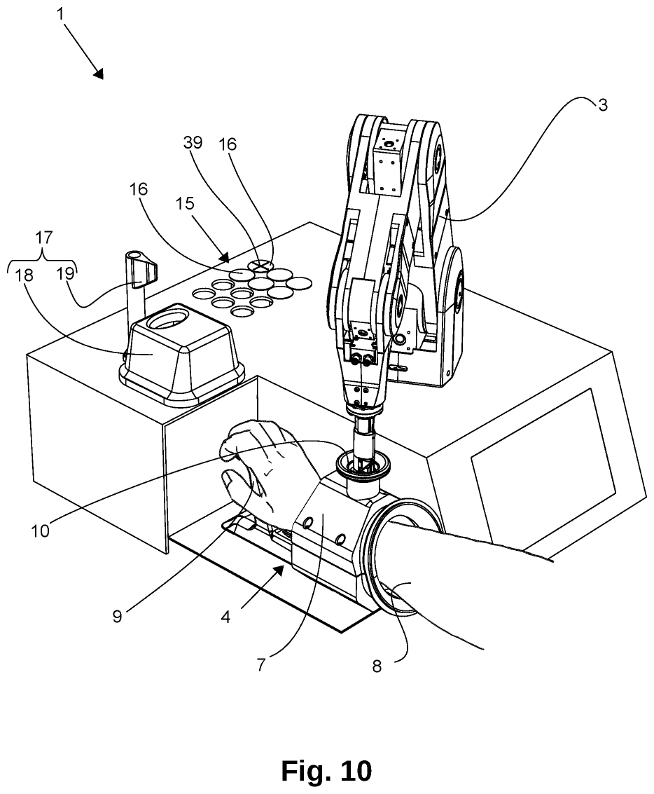

The robotic arm 3 now starts making, with the aid of the blade 30 at the bottom of the gripping element 5, a cross cut 39 in the top of the package 16 of an injector 2. This cross cut 39 can be clearly seen in FIG. 10. This cross cut 39 is possible because the end of the robotic arm 3 can rotate about a vertical geometric axis A5, such that the blade 30 can make two cuts in mutual perpendicular directions.

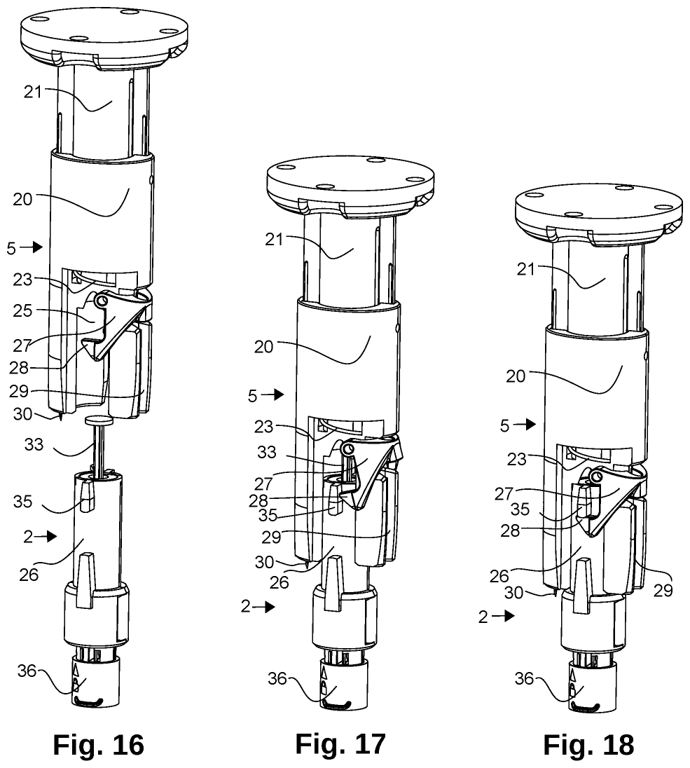

The robotic arm 3 subsequently lowers the gripping element 5 into the package 16, first just above the filled and ready-to-use injector 2 located in the package 16, as shown in FIG. 16, and subsequently over the injector 2.

Herein, the hook element 27 is pushed aside against its spring tension, as shown in FIG. 17, until the hooks 28 of the hook element 27 are located under the projections 35 on the housing 26 of the injector 2, after which the hook element 27 returns again to its original position due to its spring tension, as shown in FIG. 18.

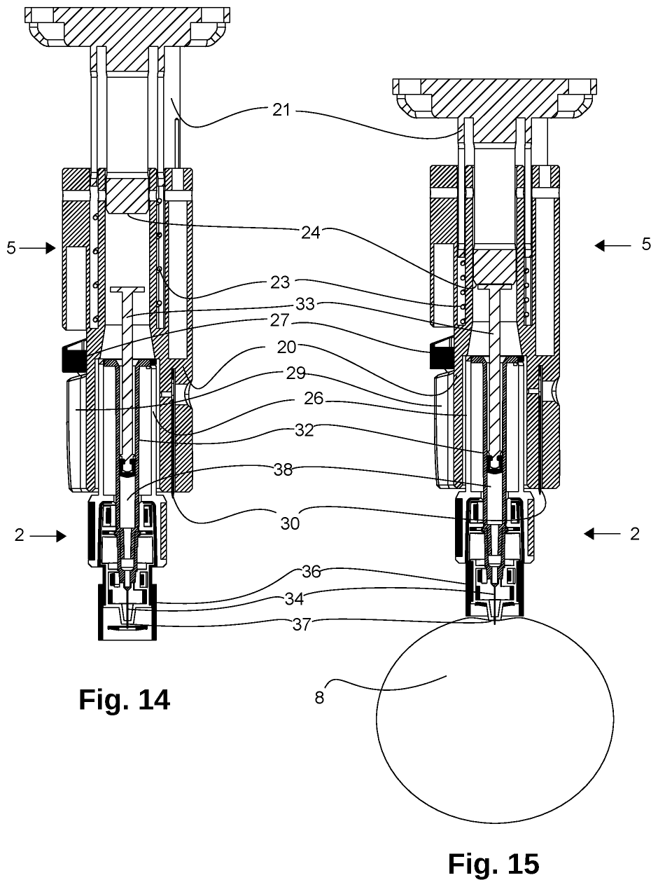

The injector 2 is now located in the receiving cavity 25 of the first component 20 of the gripping element 5, wherein the injector 2 is held in place by the hooks 28 of the hooking element 27. The now obtained state of the device 1 is shown in FIG. 9, while the now obtained state of the gripping element 5 and the injector 2 are shown in cross section in FIG. 14.

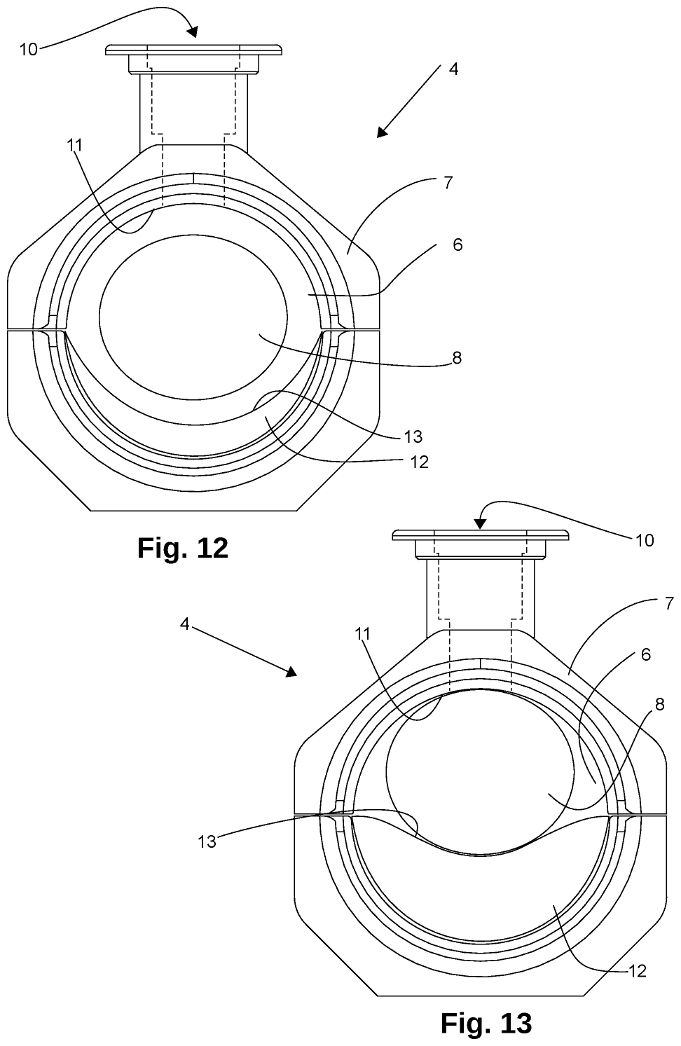

Subsequently, the air cushion 12 is inflated, such that the person's lower arm 8 is pushed upwards by the air cushion 12 and is clamped between the tunnel body 7, in particular the upper part 11 of its inner surface, and the air cushion 12, in particular the upper surface 13 thereof. The state of the forearm 8 of the person before and after, respectively, inflation of the air cushion 12 is shown in cross section in FIGS. 12 and 13, respectively. Due to the air cushion 12, the forearm 8 of the person is clamped in a mild, non-painful and harmless way.

Subsequently, the end of the robotic arm 3 is moved towards the arm holder 4, until the injector 2 is located above the passage 10 in the tunnel body 7. The end of the robotic arm 3 is now slowly lowered. As a result, the protective cap 36 of the injector 2 is placed on the skin of the forearm 8 of the person.

The downward movement of the end of the robotic arm 3 is continued, such that the housing 26 of the injector 2 is partially pushed into the protective cap 36 and slides in there, such that finally the injection needle 34 extends from the protective cap 36 and penetrates the skin of the forearm 8. The injector 2 is now in a state of use. In this state of use, the protective cap 36 and the housing 26 are also shifted relative to each other by their maximum distance.

During the aforementioned downward movement of the end of the robotic arm 3, the first component 20 and the second component 21 of the gripping element 5 remain in the same mutual position relative to each other thanks to the push spring 23.

Since the flat surface 37 of the lower part of the housing 26 of the injector 2 acts as an abutment for the skin surrounding the point of penetration of the injection needle 34 into the skin, the injection needle 34 cannot penetrate deeper than the envisaged depth of 0.85 mm into the skin such that the tip of the injection needle 34 has an intradermal position.

The downward movement of the end of the robotic arm 3 is continued.

Since the housing 26 of the injector 2 cannot move further downwards, the resistance of the push spring 23 is overcome and the second component 21 of the gripping element 5 slides into the first component 20 until the abutment 24 contacts the plunger 33 of the syringe 32 and subsequently pushes the plunger 33 further downwards, such that the liquid 38 in the syringe 32 is pushed out of the injector 2 via the injection needle 34 and is injected intradermally. In FIG. 15 the gripping element 5 and the injector 2 are shown at the moment, during the downward movement, the abutment 24 comes into contact with the plunger 33.

For the sake of completeness, it is noted that the downward movement of the end of the robotic arm 3 in this example is, but not necessarily, a linear, continuous movement.

The end of the robotic arm 3 is now moved to the receiving station 17, wherein the gripping element 5 is oriented such that the end of the pin 19 lies in the guide groove 29 in the gripping element 5. The end of the robotic arm 3 is now slowly moved downwards, whereby the end of the pin 19 pushes against an abutment on the hook element 27 and causes the hook element 27 to rotate against the spring tension such that the hooks 28 are removed from under the projections 35 on the housing 26 of the injector 2.

As a result, the used injector 2 will fall from the gripping element 5 due to gravity and end up in the receptacle 18 for safe processing.

The injection is now finished. A valve in communication with the air cushion 12 is now opened such that the air flows out of the air cushion 12 and the person can remove his arm from the arm holder 4. The device 1 is now ready to perform a next injection.

Of course, the above-described device 1 and injector 2 may also be used for subcutaneous injections, in particular by increasing the distance by which the injection needle 34 projects from the lower part of the housing 26 to a distance of between 1.2 and 5.0 mm.

Although a right arm is shown in the figures, it is obvious that both the left arm and the right arm of the person can be placed in the arm holder 4 and used to administer the injection.

1-23. (canceled)

24. A device for administering a liquid to a human by means of injection of the liquid, the device comprising a movement device and a gripping element for gripping an injector, the injector comprising an individual dose of the liquid and an injection needle, wherein the movement device is arranged to move the gripping element, wherein the injector is an injector comprising a housing and comprising an operating member to cause the liquid to move out of the injector via the injection needle, the gripping element being provided with a first component for engaging the housing, the gripping element being provided with a second component for operating the operating member, wherein the first component and the second component are slidable relative to each other, the first component being connected to the movement device via the second component.

25. The device of claim 24, wherein the device is arranged to place the injector on the limb by means of a linear movement of a movable section of the movement device and to operate the injector by continuing the linear movement of the movable section.

26. The device of claim 25, wherein the linear movement is a downward movement.

27. The device of claim 26, wherein the first component and the second component are resistively slidable relative to each other,

28. The device of claim 24, wherein the device is arranged to effect gripping an injector by the gripping element autonomously and to effect removing an injector from the gripping element autonomously.

29. A device for administering a liquid to a human by means of injection of the liquid, the device comprising a movement device and a gripping element for gripping an injector, the injector comprising an individual dose of the liquid and an injection needle, wherein the movement device is arranged to move the gripping element, wherein the device comprises a positioning unit for positioning and temporarily fixing a limb to be injected of a human body, the positioning unit comprising a first clamping member with a first surface and a second clamping member with a second surface, the second surface being movable relative to the first clamping member in a direction at least partially perpendicular to the first surface to thereby clamp the limb between the first clamping member and the second clamping member.

30. The device of claim 29, wherein the injector is an injector comprising a housing and comprising an operating member to cause the liquid to move out of the injector via the injection needle, the gripping element being provided with a first component for engaging the housing, the gripping element being provided with a second component for operating the operating member, wherein the first component and the second component are slidable relative to each other, the first component being connected to the movement device via the second component.

31. The device of claim 29, wherein the second clamping member is an air cushion.

32. The device of claim 29, wherein the positioning unit comprises a passage for letting through the injector or a part of the injector.

33. The device of claim 29, wherein the limb is an arm with a hand, the positioning unit comprising a hand rest for the hand.

34. The device of claim 24, wherein said movement device is a robotic arm, wherein said robotic arm comprises an end which is arranged to move between different positions, wherein said end is provided with said gripping element.

35. The device of claim 24, wherein said movement device is arranged to move said gripping element between different spatial positions which comprise a first position for gripping an injector by the gripping element, an injection position for operating the injector and a second position for removing the injector from the gripping element.

36. The device of claim 24, wherein the injector is an injection device, an intradermal injection device, or an intradermal delivery device.

37. The device of claim 24, further comprising at least one injector comprising an individual dose of a liquid and an injection needle.

38. A method for injecting a liquid into a human body, wherein an injector is used which comprises an individual dose of liquid to be injected and which comprises an injection needle and which comprises an operating member for causing the liquid to move out of the injector via the injection needle, wherein the injector is picked up by a device, the device comprising:
a movement device and a gripping element for gripping an injector, the injector comprising an individual dose of the liquid and an injection needle, wherein the movement device is arranged to move the gripping element, wherein the injector is an injector comprising a housing and comprising an operating member to cause the liquid to move out of the injector via the injection needle, the gripping element being provided with a first component for engaging the housing, the gripping element being provided with a second component for operating the operating member, wherein the first component and the second component are slidable relative to each other, the first component being connected to the movement device via the second component, and
the device is placed on a limb of a person to be injected, wherein subsequently the injection needle is inserted into the limb of the person to be injected by the device, wherein subsequently the operating member is operated by the second component,

39. The method of claim 38, wherein after the operating member has been operated, the movement device moves the injector from the limb to a position where the injector is removed from the movement device.

40. The method of claim 38, wherein the limb is temporarily fixed by means of a positioning unit before the placement step is completed.

41. The method of claim 38, wherein the device comprises a positioning unit for positioning and temporarily fixing a limb to be injected of a human body, the positioning unit comprising a first clamping member with a first surface and a second clamping member with a second surface, the second surface being movable relative to the first clamping member in a direction at least partially perpendicular to the first surface to thereby clamp the limb between the first clamping member and the second clamping member.

42. The method of claim 38, wherein said injector is an injection device, an intradermal injection device, or an intradermal delivery device.
